(19) United States
(12) Patent Application Publication
(10) Pub. No.: US 2007/0135676 A1
(43) Pub. Date: Jun. 14, 2007

(54) Method and apparatus of obtaining peace of mind

(76) Inventor: Hoton How, Belmont, MA (US)

Correspondence Address: Hoton How, 262 Clifton St., Belmont, MA, 02478, US

(21) Appl. No.: 11/657,311

(22) Filed: Jan. 25, 2007

Related U.S. Application Data
(63) Continuation-in-part of application No. 11/175,593, filed on Jul. 7, 2005.

(51) Int. Cl. A61M 21/00

(52) U.S. Cl. 600/26

(57) ABSTRACT

Disclosed are one method and one apparatus which teach techniques to obtain peace of mind. It is believed that quantum entanglement from this physical world can carry information which might be vulnerable to a human's quantum mind especially when one is performing meditation, exercising Qi-Gong, taking rest or relaxation, or at sleep. Protection is thus needed to protect a quantum mind during these mentally weakened situations.

### CROSS REFERENCE TO RELATED APPLICATIONS

This is a continuation-in-part of application Ser. No. 11/175,593 filed Jul. 7, 2005, now U.S. Pat. No. ______, granted ______.

### FEDERALLY SPONSORED RESEARCH

(Not Applicable)

### SEQUENCE LISTING OR PROGRAM

(Not Applicable)

### BACKGROUND OF INVENTION

1. Field of Invention

This invention is directed to one method and one apparatus to obtain peace of mind. More specifically, this invention teaches to use a magnetically suspended enclosure to effectively isolate quantum entanglement from invading the human body and thereby obtain peace of mind.

2. Prior Art

The modern person is constantly suffering from stress. As such, sleeping and anti-depression pills are commonly dosed by modern persons these days in order to overcome mental issues related to anxieties, fears, depressions and so forth. The modern person is in need of shelter protecting subconscious feelings against external disurbances to provide peace of mind. The prior art is not able to satisfy this modern person's need.

### OBJECTS AND ADVANTAGES

Accordingly, it is an object of the invention to address one or more of the foregoing disadvantages or drawbacks of the prior art, and to provide such an improved method and apparatus to obtain peace of mind. It is recognized that external spiritual disturbances are coded with quantum-entangled waves. Therefore by blocking external quantum entanglement from entering the body of a human being, external spiritual disturbances can be effectively minimized, if not totally eliminated, thereby obtaining peace of mind.

Other objects will be apparent to one of ordinary skill, in light of the following disclosure, including the claims.

### SUMMARY

In one aspect, the invention provides a method enabling peace of mind to be obtained by isolating the body of a human being from the earth environment quantum mechanically. As such, external quantum entanglement is disabled or weakened in coupling with the human body and hence external spiritual disturbances are effectively minimized, if not totally eliminated.

In another aspect, the invention provides an apparatus enabling peace of mind to be obtained by isolating the body of a human being from the earth environment quantum mechanically. As such, external quantum entanglement is disabled or weakened in coupling with the human body and hence external spiritual disturbances are effectively minimized, if not totally eliminated.

### DRAWINGS

Figure

For a more complete understanding of the nature and objectives of the present invention, reference is to be made to the following detailed description and accompanying drawings, which, though not to scale, illustrate the principles of the invention, and in which:

FIG. 1 shows one example of quantum entanglement that a man-made signal is coded with an electron beam and a positron beam resulted from pair-creation from a gamma beam.

FIG. 2 shows a redrawn pattern of the cross-sectional view of the axoneme of an actinosphaerium, which is a tiny urchin present at the early Cambrian explosion. In FIG. 2 the circles represent microtubules, and the whole picture resembles exactly a broadband dipole spiral antenna commonly employed by a wireless microstrip circuit, as shown in FIG. 3.

FIG. 3 shows a broadband dipole spiral antenna commonly employed by a wireless microstrip circuit, which resembles exactly the microtubule pattern shown in FIG. 2.

FIG. 4 shows the learning process of a paramecium to avoid an obstacle. Without an eye and a brain a paramecium, which is a single-celled organism, can “see” an obstacle and “learn” skills to avoid it, as if it were a multi-celled organism possessing an eye and a brain.

FIG. 5 shows the 9+2 pattern of microtubules in cross section of a cilium observed under an electron microscope, left, revealing the similarity to a dipole loop antenna used for TV UHF reception, right.

FIG. 6 shows a bundle of microtubules appearing with a flagellum forming a monopole antenna. The cross-sectional view of this bundle of microtubules is shown in FIG. 5, left, which forms a dipole loop antenna.

FIG. 7 shows cell mitosis that centrosomes and chromosomes are duplicated first and then pulled apart by spindle fibers before the cell is finally split.

FIG. 8 shows partially the fertilization process that the sperm cell is entering an egg cell and the tail of the sperm cell, which acts like a monopole antenna, attracts a spirit signal. After entering the egg cell, DNAs from both of the parent cells join together to form new chromosomes for the child, and the child's spirit resides at the newly reformed centrosomes constructed from the sperm's tail.

FIG. 9 shows that Trojan-horse codes can invade into a PC via whatever a path, wireless connection or disk sharing, etc., thereby taking over the control causing disasters.

FIG. 10 shows one example of the preferred embodiment of the invention that a human being is resting or meditating in an isolated closet which is magnetically levitated above the floor ground so as to effectively cut off quantum entanglement from this physical world.

### REFERENCES NUMERALS

201 A Human Being
202 Mattress/Pad
203 Empty Space
204 Floor Ground
211 Inner Wall (Sealed Air Tight)
212 Outer Wall (Sealed Air Tight)

### DETAILED DESCRIPTION

### Background

Nowadays there is little doubt about the validity of quantum mechanics. Quantum entanglement can carry information, and this argument was used by John Bell in 1964 to resolve the EPR (Einstein, Podolsky, and Rosen) paradox. Today simple quantum computers have already been fabricated and demonstrated invoking computations on quantum entangled phases or states (for example, Chuang, Laflamme & Yamamoto, 1995). Quantum computations are performed onto qubits, as in contrast to classical computations for which digital bits are processed sequentially and/or parallelly in the CPU module. Via processing qubits, quantum computations are effectively implemented in the Hilbert space constructed from the space-time frame, either partially or entirely, and the algorithms take the form of Feynman path integrals, for example. However, on interacting with a classical measurement apparatus, quantum computations inevitably collapse causing the involved entanglement in quantum states to lose coherency. This decoherencing encounter has prevented a large quantum computer from being physically constructed. With regard to Feynman's path integral calculations, quantum decoherency results in path selection that a specific path is selected among all of the possible paths involved in Feynman path integrals, and the selected path may or may not represent a physical one. Because people are not familiar with Feynman path integrals, the quantum world has appeared weird and irrational. More of these will be discussed later, such as the “Orch OR” and the many-worlds picture.

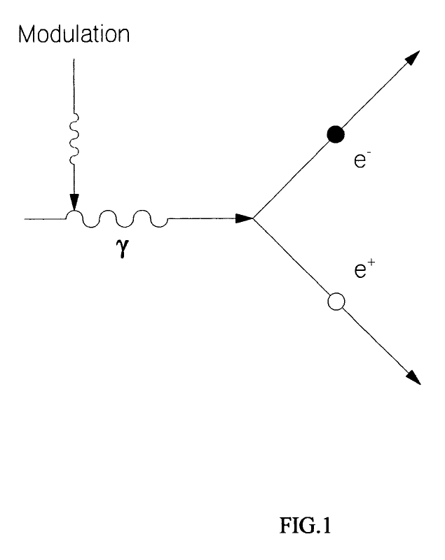

Quantum information, or quantum entanglement is illustrated in FIG. 1, which represents a primitive example that classical phases of photons are converted into classical phases of electrons. This is in contrast to the general case that quantum phases of photons are converted into quantum phases of electrons. A classical phase comprises a definite phase value, and a quantum phase means the phase whose value is indefinite subject to a probability distribution function. A classical phase is a special case of a quantum phase wherein the probability shows a delta-function-like distribution. Man-made information implies classical phases, and conscious information, for example, belongs to quantum phases. In FIG. 1 a series of gamma photons are phase-modulated to collaboratively carry man-made information, in a manner analogous to their low-frequency counterpart that IR photons jointly carry an Internet message, for example. Of course, in FIG. 1 the photons can carry quantum phases, but then it is less likely that quantum phases are produced via a classical setup. Let these gamma photons be of sufficient energies so that they decompose into electron-positron pairs (in the presence of matter). After decomposition the original information, whether classical or quantum, or both, residing on the photon phases does not diminish; rather it is passed over to the created pairs of electrons and positrons. Assume that the electrons (and the positrons) are well separated in distance so that they are considered as non-interacting particles. But these electrons (and positrons) are now correlated in their respective phases forming entanglement thereby carrying information. Phases of this kind are conventionally ignored by physicists, because they do not possess a physical meaning after the wave functions are normalized, from the “energy” point of view. However, these phases in wave functions can entangle to collectively carry information like spirits, from the “nonphysical” point of view, as evidenced by FIG. 1. Materialists and evolutionists always emphasize on the energy part of a phenomenon, and psychists and Platonists are more concerned in the nonphysical part of this world. Quantum mechanics entails them all.

In our physical world there are enormously many non-interacting quantum systems allowing quantum entanglement to occur, in a manner analogous to FIG. 1. For example, the inner shells of electrons in atoms or in molecules which do not interact among one another energetically, but their individual phases are apt to be entangled collectively thereby serving as a storage medium for quantum information. The content of this information-storage medium permeating our physical world thus formed is enormously huge, and it is effective and secure: it will never lose coherency in the constituent phases of the world's quantum systems, since classical measurements are performed with respect to a bundle of quantum transducers intermediating this quantum storage medium, and they are not performed directly onto the medium itself Upon classical measurements, quantum entanglement across a quantum transducer is corrupted, but not the world's storage medium. These quantum transducers will later be identified as microtubule lattices, which are known as biological quantum computers, and classical measurements on microtubules will ultimately bring in consciousness and unconsciousness. Although human beings are not able to fabricate large quantum computers, the biological species have done so billions of years ago. This information storage medium of our physical world forms a quantum hologram as a whole, and information thus embedded and entangled will be distributed nonlocally over space and time showing phase coherency, and Lorentz symmetry is attained as implied by the fundamental physics law.

On reading information reserving phase coherency one needs to employ a periodic structure. For example, many radiators are deployed at regular spatial locations to form a phased array antenna in transmitting/receiving coherent electromagnetic signals. In retrieving the 3D image of a scene from an optical hologram a reference laser beam is required to reference phase relationship thus to enable the stored interference pattern to be reconstructed, i.e., the reference beam provides spatial periodicity to allow a phase reference to be established. In the biological world microtubules are universally found over all species in which tubulin proteins are stacked periodically into tubes assuming a rigorous translational symmetry, suggesting that they may be coherent phase detectors or transducers of the nature. Otherwise, it is not understood, because the translational symmetry means entropy is minimized and hence it violates the thermodynamic law. There must be a reason that the nature dictates all the biological species to accommodate such a universal and unnatural but energy-expensive structure of a microtubule, regardless the species is a prokaryotic cell or a eukaryotic cell, a single-celled organism or a multi-celled organism, a plant or an animal. The reason is that the nature wants all the earth lives to be equally eligible to access the information database contained with the quantum hologram of our physical world just described, either contributing data to it, or retrieving data from it, along the course of evolution as a whole.

Microtubules are mainly found in three different kinds of biological organelles and they are centrosomes, cilia, and nerve fibers. These organelles are separately discussed. Today most authors believe our brains are quantum computers, and conscious and unconscious experiences are aroused whenever phase decoherency occurs along the nerve fibers forming a neural network. Hameroff has proposed the “Orch OR” picture to portray the process of quantum consciousness wherein he assumed the tubulin proteins appearing with a microtubule act as qubits. What is missing in the present scenario about a quantum brain is the storage medium holding memories consisting of events or paths either physically happened or fictitiously imagined. Here it is believed the storage medium rests on the glial cells which outnumber neurons by about 10 to 1. The glial cells feed data onto or retrieve data from nerve fibers in a coherent manner so that no quantum decoherency occurs; when conscious/unconscious is aroused, quantum decoherency occurs thereby destroying the information content allocated with the neural network, but it leaves the glial cells intact not to risk losing memories at all. Although glial cells in a quantum brain store personal information, it also connects to the universal database of our physical world via entanglement so that other persons might be able to read and control a quantum mind via telepathy, hypnosis, pray, or curse, for example.

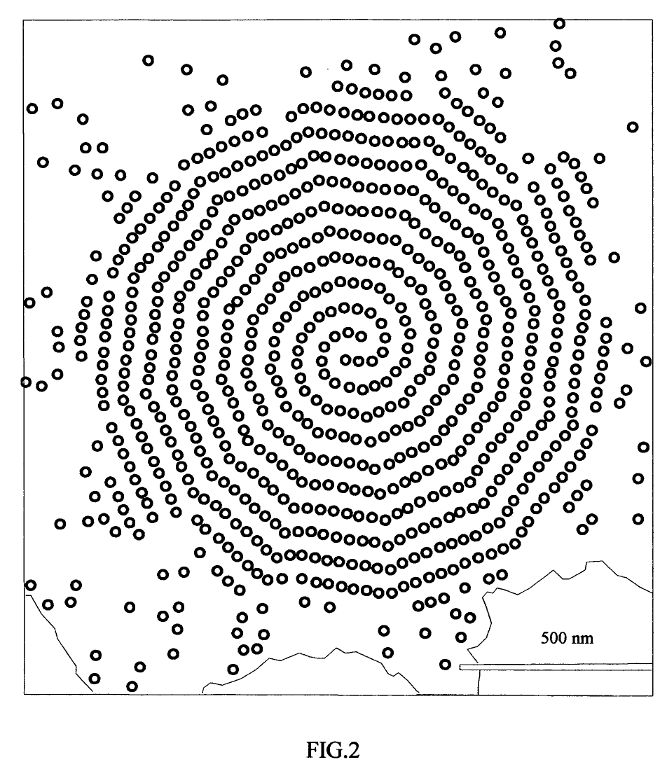

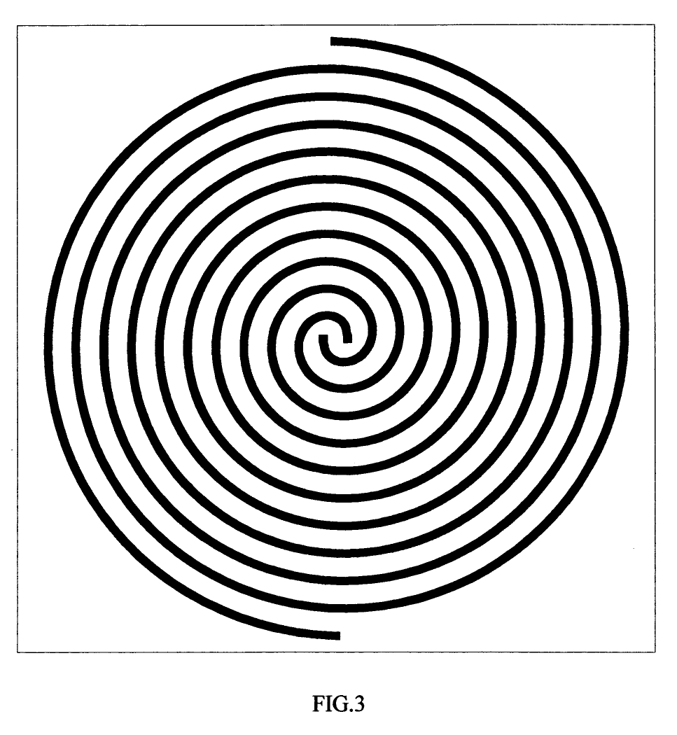

By using “Orch OR” Hameroff pointed out that tiny urchins, worms, and suctorians present at the early Cambrian explosion might have developed consciousness, which admitted advantages to species survival and hence accelerated evolution. FIG. 2 was redraw from L. E. Roth to show the cross-sectional view of the axoneme of an actinosphaerium, which is a tiny urchin present at the early Cambrian explosion. In FIG. 2 the circles represent microtubules and the whole picture resembles exactly a broadband dipole spiral antenna commonly employed by a wireless microstrip circuit, as shown in FIG. 3. This confirms the aforementioned statement that microtubules with tubulin proteins distributed regularly in space acting as the qubit transducer elements serve the phased-array antenna functions on receiving/transmitting coherent quantum information in broadband over the universal database of our physical world. Quantum entanglement takes place at an infinite velocity. However, to perceive or realize a message concealed with quantum information by employing a classical apparatus, namely microtubules, the message cannot propagate faster than the speed of light. Like photons, spiritual or biological messages do not involve energy, and hence they must propagate at the light velocity. From FIG. 2 it is seen that the spiral antenna of an actinosphaerium is operational at the wavelength from 0.3 to 3 μm, corresponding to the frequency spectrum from 1 to 10×10¹⁴ Hz. Remarkably, this frequency range covers the visible light, which is from 4 to 7.5×10¹⁴ Hz. Thus, an actinosphaerium can “see” like human eyes, and better. The visual bandwidth of a human being is 1:2, whereas for an actinosphaerium it is 1:10. Thus, an actinosphaerium see more colors than us.

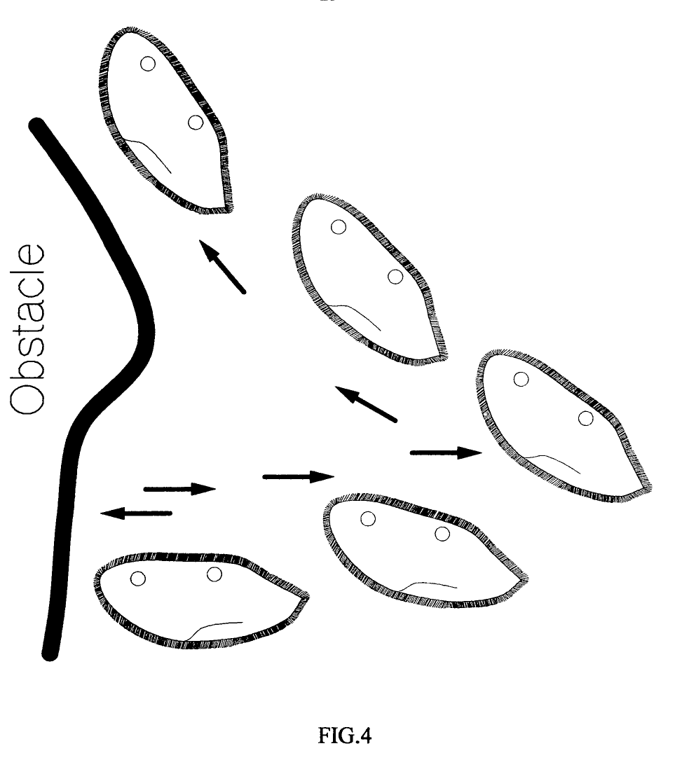

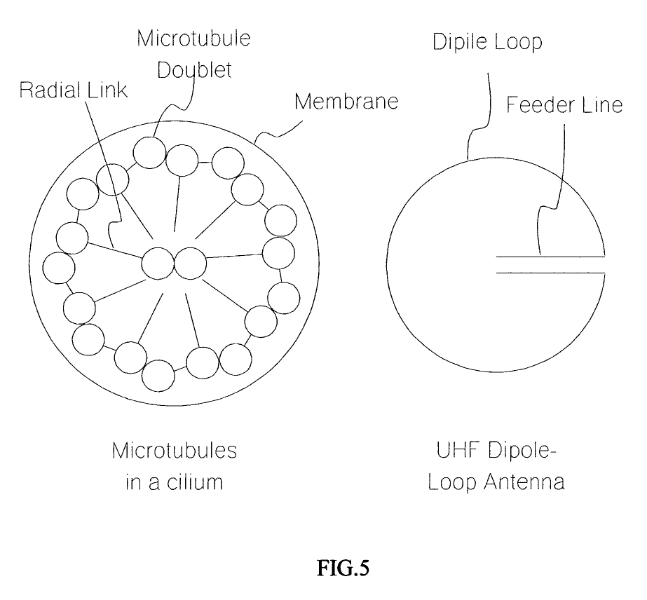

Without an eye and a brain a paramecium, which is a single-celled organism, can “see” an obstacle and “learn” skills to avoid it, as if it were a multi-celled organism possessing an eye and a brain. Paramecium's learning process is shown in FIG. 4. Like a tiny urchin just described, a paramecium has developed consciousness and it has the ability to perceive. That is, a paramecium can use its cilia to transmit and receive quantum information accessing the universal database of our physical world thereby recognizing the existence of an obstacle; consciousness is subsequently aroused via quantum computation taking place along the bundles of microtubules again in the cilia in a manner analogous to “Orch OR”. From FIG. 5 below, a paramecium “sees” an obstacle with blue color, since the dipole antenna of a cilium is operational at 4.4×10¹⁴ Hz.

Seen under an electron microscope, a cilium shows a 9+2 pattern of microtubules in cross section revealing the similarity to a dipole loop antenna used for TV UHF reception. This is shown in FIG. 5 wherein the 9 microtubule doublets form a dipole loop with the 9 radial links acting as feeder lines. The only difference between these two structures is that for the UHF antenna the feeder lines are allocated at the beginning and the end terminals of the loop, whereas for the cilium radial links are distributed evenly across the loop's angular positions. This enables the cilium to see things in monochromic “blue” color. In order to learn skills thus to memorize past lessons a paramecium must have self-memories, and this local storage medium must locate in the vicinity surrounding the microtubules. It is believed that the memories are allocated with those cytoplasmic fibers named intermediate filaments. Thus, the cilia as a whole function like a mini-brain capable of executing quantum computation in a miniaturized fashion barely enough to bring about consciousness, as argued by Hameroff.

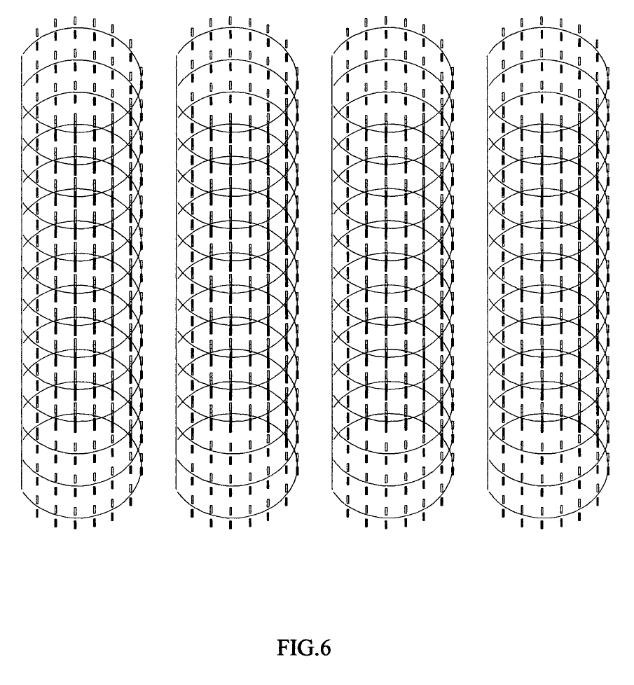

The structure of a flagellum is identical to a cilium except the former has a longer length. Microtubules forming a flagellum act as a monopole antenna, similar to the long-arm antenna used for TV VHF reception, as shown in FIG. 6. In FIG. 6 a tubulin dimer is schematically shown by two arms one colored white and the other black to simulate a conventional dipole radiator. In fact tubulin dimers collectively serve as a qubit processor, as pointed out by Hameroff. A sperm cell has a flagellum tail whose importance will become clear shortly. It is believed that the life of a biological cell evolves adapting two sets of instructions: a hardware recipe and a software spirit. While the recipe is identified as the DNA sequences coded with the chromosomes, the spirit resides at the centrosomes of the cell, or more precisely, it is concealed with the pericentriolar material (PCM) intermediated by centrioles. The centrosome consists of a pair of mutually perpendicular centrioles assuming a 9+3 microtubule pattern, which is similar to the 9+2 microtubule pattern of a cilium shown in FIG. 5 except the doublets are replaced by the triplets. Thus, the centrioles are able to assess the spiritual information stored with PCMs thereby inspiring and monitoring a life cycle to start and to evolve. This process is analogous to the brain function that nerve fibers intermediate personal memories stored with the glial cells, but in a miniaturized fashion. Due to the small mass of one or two centriole, quantum computations occurring within one single cell may not arouse consciousness, according to “Orch OR”. However, consciousness can arrive if body cells are linked together as a whole via practicing Qi-Gong or meditation, for example. This is especially true that centrioles entangle with nerve fibers quantum mechanically both decohering at the α-brain wave frequencies, as will be discussed shortly.

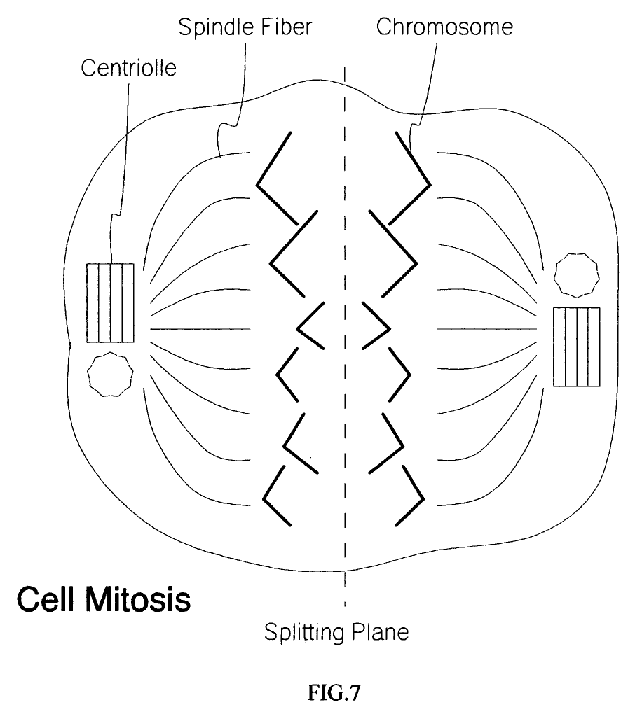

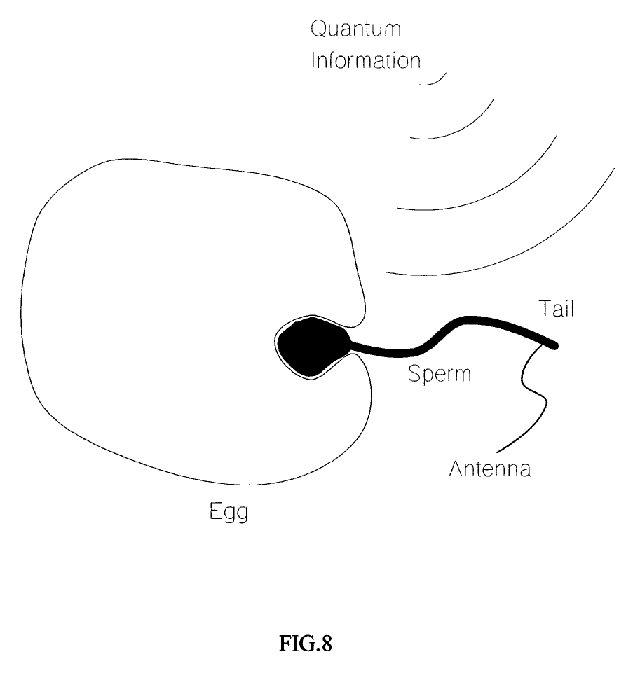

It is known that during cell mitosis centrosomes and chromosomes are duplicated which are pulled apart by spindle fibers before the cell is finally split, as shown in FIG. 7. However, during cell meiosis centrosomes are first destroyed and chromosomes are not duplicated, and, as a consequence, the sex cells have only one half the chromosomes and they do not have centrosomes. It was seen under an electron microscope that after fertilization chromosomes from the egg and the sperm cells join together thereby retaining back the chromosomes, and the centrosomes of the zygote cell are newly reformed from the sperm's tail. The fertilization process is partially shown in FIG. 8. In FIG. 8 it is seen the sperm's tail acts like a monopole antenna, and it attracts and receives a spiritual soul before entering the egg cell; after entered the egg cell, the sperm cell with a spirit inhabited becomes the new centrosomes surrounded by PCMs. Thus, on growing up, while the child's physical body is gradually built according to the joint recipes of the combined DNA sequences of his parents, and the traits or characters of the child are inhered from an extraneous spiritual source or soul signal transplanted during fertilization. The spirit of the child has nothing to do with the parents, but rather imposing a possible causal relationship or linkage.

The twins can show remarkable similarity both in outside appearance and in inside traits. This is because they are built according to identical hardware recipes, the DNA sequences, as well as the software information, the spiritual content; they may encounter similar fates during their this-life cycles, because they share the same causal relationship or linkage succeeded from their same-previous live cycles. Most noticeably, they communicate with each other in a seemly extraordinary way beyond normal understanding, and they are moved under the same emotional mood even if they are separated in distance. In fact they are tightly bound via quantum entanglement of this physical world never weakened by the finite velocity of light. A quantum entangled state is similar to a superconducting state, both of which show long-range coherency in the macroscopic scale. Thus, by cutting apart a superconductor ring the supercurrent stops flowing instantly no need to wait for the causal signal to propagate at the finite speed of light, as in contrast to the case of a normal conductor ring wherein no signal can propagation along it faster than the light speed.

It is commonly believed one soul can be transmigrated from one life cycle into another. In Tibet a dying buddha foresees his reincarnation at a specific space-time location. There are many famous predictions about the future evolvement of our secular world written in ancient books, and, as time goes by, many of these predictions have been proven true. Quantum entanglement has no direction in time, since it is massless. According to Heisenburg uncertainty principle, when mass goes to zero, the uncertainty in time becomes infinite: the future is equivalent to the past from the quantum mechanical point of view. In fact, the time scale is set by classical measurements by which quantum decoherency occurs as a consequence of consciousness thereby causing quantum entanglement to collapse, as pointed out by Hameroff. If conscious is suppressed, future becomes predictable.

However, all the predictions about the future made by those great authors are ambiguous in context. This is because future is not clearly defined. Like the formulation of Feynman's path-integral, which serves as the foundation of the quantum field theory, the transition probability from one space-time location to another involves integration over all paths connecting these two locations, and the paths can be physical or nonphysical. A classical path can only result if the quantity of mass multiplied by time, which is called action, is much greater than the Planck constant. Thus, if the person, who ever made predictions about the future, still showed a minimum amount of consciousness in mind, namely still performing any of the classical measurements invoking masses or energies in mind, he cannot make predictions far into the truth of the future: the more consciousness involved, the more ambiguous and less accurate about his prediction.

Thus, the universal database of our physical world contains phase information encompassing all paths from the past to the future, true or false, physical or fictitious, forming a giant quantum hologram This is in contrast to a classical picture that a magnetic recording tap, for example, contains data bits 1 and 0. In contrast the quantum storage are in unit of qubits which are much more informative than digital bits. In view of quantum phases discussed previously, it lays the many-worlds picture, which was proposed by Hugh Everett III in 1957, that the universe contains nothing but illusions, not just one, but inconsistently many. This explains the seemly contradictory feature of divination that an unrealized future, or a false future, can act back the truth of the present. Though weird, it is true.

Quantum entanglement can only happen in the presence of matter. Thus, quantum information will congregate more in solid and in water than in air. This is the reason that a dying patient feels his soul leave his body lying straightly atop on the ceiling looking downward onto the doctors rescuing his life, depicting a common picture of the near-death experiences. That is, the soul prefers to attach to a piece of solid (or liquid) than just fly empty in air. The reason that a dying person has access to his soul's feeling thereby perceiving its activity is due to the low oxygen-concentration in blood flowing into the brain, as described in the following paragraph.

During sleep the brain waves measured with EEG (electroencephalogram) electrodes have shown that sleep cycles each takes about 2 hours. The β(15-40 cps) and α (9-14 cps) waves are observed during wakeup, and θ (5-8 cps) and δ (1.5-4 cps) waves are observed during shallow and deep sleeps, respectively. In addition there is REM (Rapid Eye Movement) period occurring during sleep prior to wakeup whose brain waves are the β-type. The α-waves occur during the high-relaxation stage, such as on performing meditation or practicing Qi-Gong thereby obtaining peace of mind. Dreams made during REM are close to reality whose contents may be recalled after wakeup, whereas dreams made with the θ and δ brain waves can hardly be memorized. The β-waves are believed to be associated with memory access onto the glial cells of the brain activating consciousness via classical measurements performed by nerve fibers at a decoherencing rate of 15 cps and higher; the α-waves relate to access of the quantum information contained with the universal database of our physical world activating consciousness via classical measurements also performed by nerve fibers at lower decoherencing rates of 9-14 cps. Even lower decoherencing rates are exercised by the θ and δ waves accessing the information content of PCMs via centrioles contained with the body cells. Centrioles can link nerve fibers thereby tuning unconscious into conscious. This allows deep unconsciousness to be explored consciously revealing entanglement in causality in past and in future life cycles. A hypnotist may enter a patient's unconscious world via deep hypnosis.

The decoherencing rates, or the rates of performing classical measurements onto microtubules allocated with nerve fibers, are determined by the oxygen concentration in blood entering the brain. During wakeup the oxygen concentration is high, and hence personal consciousnesses are exclusively aroused. When deeply relaxed at meditation, for example, the breathing rates are reduced and the oxygen concentration is lowered in the brain. When this happens, the soul begins to step out from the body confinement wandering about this physical world through quantum entanglement. Parapsychology happens as a consequence, such as telepathy, clairvoyance, precognition, remote viewing, etc. The near-death experiences described previously also fall into this category. As pointed out by Hameroff, some chemicals, such as anesthesia and psychedelic drugs, are effective in influencing the quantum decoherencing rates, similar to oxygen concentration, and hence they can alter the brain-wave types thereby accessing the different parts of this same quantum world. Lower decoherencing rates associated with θ and δ brain waves occur at even lower oxygen concentrations.

Brain function is interpreted as follows. The glial cells store past conscious experiences which are used as templates in projecting out or filtering out a classical path when quantum decoherency occurs, and quantum computations involve evaluation of Feynman's path integrals exerted upon qubits allocated with nerve fibers. Analogous to nerve fibers, quantum computations occur to cilia and centrioles, and intermediate filaments and PCMs play the same role as glial cells storing conscious and unconscious information, respectively. Note that a classical computer processing digital bits is far less efficient in calculating Feynman's path integrals and only can a quantum computer qualify for this challenging task. According to “Orch OR”, more mass, or more qubits, is involved at a higher decoherencing rate, resulting in better accuracy in selecting a classical path. Thus, during wakeup on performing an arduous work requiring concentration the brain waves show high frequency oscillations thereby warranting the work to be done with precision. At relaxing time the decoherencing rate slows down showing slower brain-wave oscillations, less qubits are involved in quantum computation, resulting in less accuracy in selecting a classical path; fictitiousness arises, giving rise to fantasies and aha experiences.

Most importantly, consciousness is based upon the process of projection or filtration, similar to the task of coherent wavelet transformation. For example, no matter how hard to describe to a color-blind person what the meanings of colors are, such as red renders anger and blue lends sadness, etc., he or she will never understand. Nobody call tell what a 4-dimensional space look like, because none of us have ever had such an experience. We can imagine and invent something new, but still these new things must be connected with our past conscious experiences. In our dreams we always see someone we already knew, even if the memory has faded out and cannot be recalled consciously. Under deep hypnosis, a patient may talk in a different dialect, because such a language has been stored in the PCMs of his body cells. The dogs and human beings both have access to the universal database of our physical world, but we cannot dream what the dogs dream, because we do not have their templates (maybe our PCMs do).

Dao and Qi-Gong have been popularly and constantly believed and practiced by Chinese people for thousands of years. Chinese people realize there are two kinds of Dao: one fills in the entire universe, called Big Dao, and the other fills in an individual's body of all species, called Small Dao, including a cow, a bird, a fish, a bug, a grass, and so on. One must keep his Small Dao permeable and clean in order to be healthy and happy. The Big Dao rules everything: it moves the sun and the moon, and it keeps the creatures alive. The purpose of a human's life is to join his Small Dao with the universe's Big Dao, and if so, his physical body will live forever. In order to achieve this purpose Qi-Gong is thus practiced, taking the form of meditation and exercising. When Qi-Gong is practiced, a stream of air or gas, which is called Qi is driven to flow inside a human body, which can be led outside to realize parapsychology. From the above statements, the Big Dao may be identified as the universal database of our physical world and the Small Dao is our body's nervous system, including the brain, the spiral cord, and the peripheral nervous system. When Qi is circulating, microtubules in the corresponding area of our body are linked together performing “Orch OR”. Qi-Gong is unconscious, or nearly conscious, because the involved mass in “Orch OR” is not sufficient, or at critical.

It is believed that group communication among all the members of the same species is needed in order to negotiate a unified pace on evolution. Negotiation takes place in the spiritual world via linkage over individuals. It is hard to believe random mutations in gene sequences can neatly divide organisms into discrete species with distinct outlooks and traits. It is more likely evolution is a consequence of group negotiation, and the spirit signal can even dictate changes in DNA codes during the fertilization process. In a small island surrounded by water or desert, biological species are found to be quite different than in the continents, because the island species have shown little linkage or causal relationship with outsiders due to geographical isolation. Note that two isolated geographic regions behave like two disconnected pieces of quantum hologram and only the linkages between biological organisms can bring them together imposing quantum entanglement. A person can have many kids, and a spirit can have many succeeders. For the long run the genes as well as the spiritual information will mix and homogenize over a geographic region showing complex linkages or coherency in blood ties and in quantum entanglement.

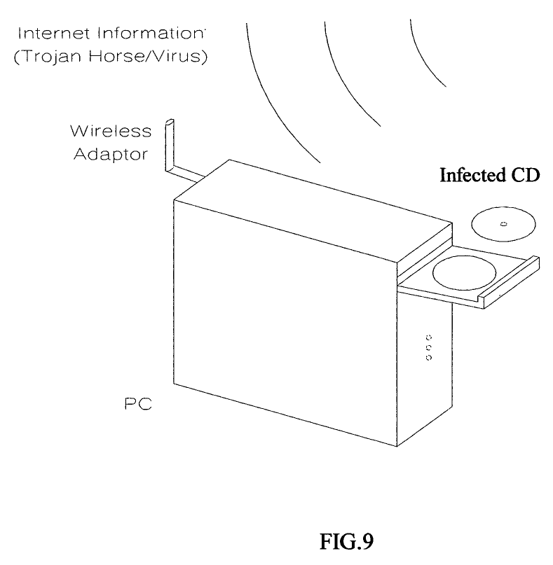

As said, our bodies are built according to hardware recipes and software spirits. The hardware part makes us deferent in outlooks and the software part makes us different in characters. This situation resembles a PC whose operation requires two parts to participate, hardware and software: the former includes CPUs, memories, and peripheral devices, and the latter Operation Systems, drivers, and applications. Our bodies are open to the outside world quantum mechanically, and there are risks we are going to be attacked and get hurt by external issues. Analogously, Trojan-horse codes can enter a PC via whatever a path, wireless connection or disk sharing, etc., thereby taking over the control causing disasters, as shown in FIG. 9. And a seemly lifeless DNA or RNA sequences appearing with an invaded pathogenic virus can even kill an elephant. An evil spirit can invade the PCMs of the body cells of a patient thereby causing his personality to split. The sounds and scenes perceived by a psychopathic patient are real from the quantum mechanical point of view, i.e., the many-worlds picture. It is also suspect that cancer cells are tuned and transitioned due to external issues like spiritual viruses causing them to lose loyalty. There is a need to protect our physical body quantum mechanically.

During wakeup, the oxygen concentration in blood is high, which warrants a quantum mind to decohere using the β-wave frequencies. When the β-decoherencing rate is used, there is no room to “listen” to the other brain waves which may hide evil messages or spirits. However, when at sleep, the decoherencing rate is lowered, giving advantages to those bad spirits letting them have opportunities to invade. It is thus desirable to block those evil spirits, especially during sleep. One effective way to do this is to temporarily isolate a person from this physical world quantum mechanically. After having stayed in the outer space for 100 days, on Oct. 18, 2005 Chinese astronomers Fei and Nie returned back to earth and announced publicly that they had no dreams in outer space for 100 days (http://news.tom.com/1020/1022/20051018-2562837.htm). This is quite peculiar, since the environment inside an orbital satellite is not much different than on earth. The only difference is that in outer space quantum entanglement with earth systems are cut off due to vacuum isolation, resulting in a much reduced information content to compose a dream. Note that by cutting half an optical hologram, for example, one still sees the original image, but with the visual intensity reduced by a factor of 4; by tearing half a photo picture, one loses half the image with the same visual intensity. In ancient China or India, a hermit exiled himself in a removed forest site, for example, to cut down mental noise or quantum entanglement in exercising spiritual pursuance. To reduce quantum entanglement, we do not need to go to outer space or a forest site, because we can realize such an isolation condition on earth at a convenient location, which is discussed in the following paragraph serving as an explicit example of the present invention.

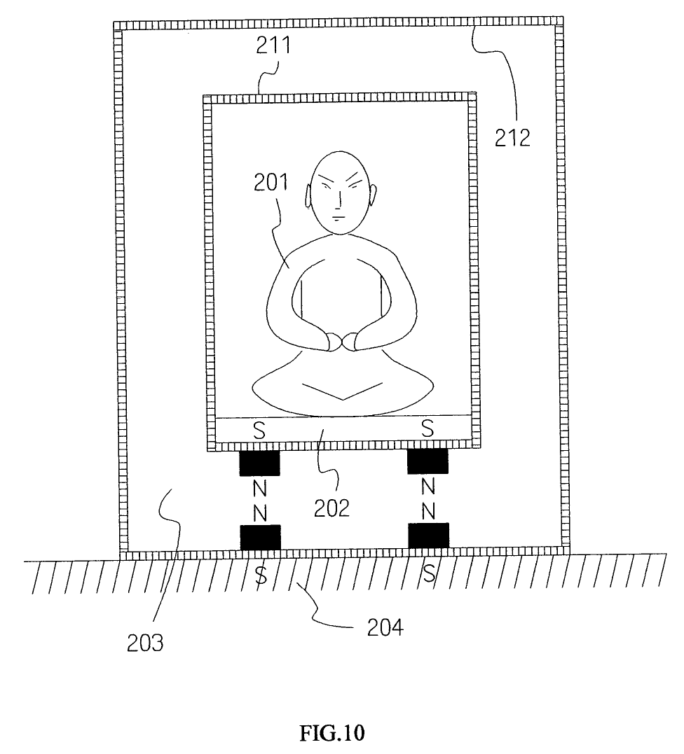

### ONE EXAMPLE OF PREFERRED EMBODIMENTS OF THE PRESENT INVENTION: FIG. 10

To illustrate the present invention one explicit example is given in FIG. 10 in which an artificial remote site is created which is effective in cutting down quantum entanglement from the rest of this physical world. In FIG. 10 Human Being 201 is sitting atop Mattress/Pad 202 taking rest or practicing meditation, for example. Mattress/Pad 202 can also be a bed so that Human Being 201 can lie down to sleep. In FIG. 10 Human Being 201 and Mattress/Pad 202 are enclosed by Inner Wall 211 defining a closet showing a sealed air-tight closure. The closet is enclosed within Outer Wall 212 which also shows a sealed air-tight closure. Inner Wall 211 is suspended within Outer Wall 212 not to make physical contact by means of magnetic lifting or levitation as shown in FIG. 10. In FIG. 10 blackened rectangular blocks denote permanent magnets whose north and south poles are marked as N and S, respectively. In fact magnets are placed at appropriate locations not necessarily to be restricted to those positions above or below the bottom surface of Outer Wall 212 or Inner Wall 211, respectively, as shown in FIG. 10. Alternatively, electromagnets or superconductor magnets, can be used substituting permanent magnets, or in combination. Empty Space 203 is shown between Inner Wall 211 and Outer Wall 212 whose atmospheric pressure is reduced so as to effectively cut off quantum entanglement from this physical world external to the closet defined by Inner Wall 211. In FIG. 10 Floor Ground 204 is shown contacting Outer Wall 212. Inner Wall 211 or Outer Wall 212, or both, is able to block electromagnetic waves and acoustic waves so as to cut down unnecessary disturbances. Air, water, or other stuff is brought in the closet defined by Inner Wall 211 to provide a comfortable stay, if deemed appropriate. This enables peace of mind to be obtained by Human Being 201 on rest, meditation, or sleep.

### CONCLUSIONS

The present invention teaches a method and an apparatus enabling peace of mind to be obtained using an artificial remote site. The site is effectively isolated from the secular world quantum mechanically, and quantum entanglement from the rest of this physical world is reduced via vacuum isolation.

I claim:

1. A method of cutting down quantum entanglement, comprising:
setting up an artificial remote site,
wherein by surrounding said artificial remote site using an empty space with reduced atmospheric pressure quantum isolation is achieved thereby cutting down quantum entanglement from the rest of this physical world.

2. The method of claim 1 wherein said artificial remote site is realized via magnetic means employing magnetic lifting and stabilizing apparatuses consisting of permanent magnets, electromagnets, superconductor-magnets, or in combination.

3. The method of claim 1 wherein air, water, or other stuff is brought in said artificial remote site to provide comfortable stay, if deemed appropriate.

4. The method of claim 1 wherein said artificial remote site is realized incorporating impermeable wall which blocks electromagnetic radiations.

5. The method of claim 1 wherein said artificial remote site is realized incorporating impermeable wall which blocks acoustic waves.

6. An artificial remote site device, comprising:
a closet in the earth environment which shows no physical contact with its surroundings,
wherein by staying inside said closet quantum entanglements can be effective cut down thereby obtaining peace of mind.

7. The artificial remote site device of claim 6 wherein said closet is magnetically levitated not showing physical contact with its said surroundings via employing permanent magnets, electromagnets, superconductor magnets, or in combination.

8. The artificial remote site device of claim 6 wherein the atmospheric pressure in the space between said closet and its said surroundings is reduced.

9. The artificial remote site device of claim 6 wherein said closet is impermeable to electromagnetic radiations.

10. The artificial remote site device of claim 6 wherein said closet is impermeable to acoustic waves.
